(19) United States
(12) Patent Application Publication
(10) Pub. No.: US 2006/0173286 A1
(43) Pub. Date: Aug. 3, 2006

(54) Method and apparatus for determining a pose of an implant

(75) Inventor: Jean-Pierre Franciscus Alexander Maria Ermes, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., Eindhoven (NL)

(21) Appl. No.: 10/538,581

(22) PCT Filed: Nov. 17, 2003
(86) PCT No.: PCT/IB03/05254

§ 371 Date: Jun. 15, 2005

(30) Foreign Application Priority Data
Dec. 18, 2002 (EP) 02080390.4

(51) Int. Cl. A61B 5/05

(52) U.S. Cl. 600/424

(57) ABSTRACT

A method for determining a pose of an implant object that is located inside a human or animal body uses a CAD model of that implant through a reconstruction X-Ray procedure that encompasses a translation-reconstruction run of the X-Ray arrangement viz ô viz the implant object. In particular, the method being comprises for an implant object that has a degree of symmetry according to an n-dimensional structure of symmetry the following. generating a first measurement configuration and a second measurement configuration regarding an X-Ray source and a prespecified implant position, and generating a first and a second implant shadow, respectively; assuming for each first and second measurement configuration an instance of the n-dimensional structure of symmetry; calculating for each of the first and second measurement configuration a pair of alternative poses of the implant object as being symmetrical with respect to the n-dimensional structure; and finding among the pairs of alternative poses two matching poses that thereby produce an angle information with respect to the n-dimensional structure of symmetry.

### BACKGROUND OF THE INVENTION

The invention relates to a method for determining a pose of an implant object that is located inside a human or animal body on the basis of a CAD model of that implant object through a reconstruction X-ray procedure, that for each measurement configuration finds a pair of alternative poses that are symmetrical with respect to said n-dimensional structure and finding a matching pose in each pair as has been recited in the preamble of claim 1. The determining of a pose, that is an abbreviation of position plus orientation of such implant object, is inter alia relevant for the purpose of checking the actual quality of the implant. For example, through wear, loosening or worse, this actual quality may have deteriorated to such effect that additional measures would be mandatory. Now, when such CAD model of the implant object is known, the pose of the implant with respect to an X-Ray imaging plane can be estimated. This imaging plane can for example be an X-Ray film or a sensing plane of an image intensifier device.

In many cases, the solution so determined for the pose is unique. However, in the case that the implant has a particular degree of symmetry, the solution is not necessarily unique. Relevant cases of such symmetry are mirror symmetry, such as is the case for certain artificial junctions, and rotation symmetry, such as for various implant objects used inside blood vessels and the like, and also, more or less “round” elements such as the “head” of an artificial hip joint. Other degrees of symmetry, such as triple (120°) or fourfold (two mirror planes at 90° with respect to each other) are less relevant, but could be applicable in certain circumstances as well. The inventor has recognized that in case of a mirror symmetry, the “shadow” of the object would be invariant under a mirroring transform of the implant object. The real cause of the problem is that a narrow-beam-generated X-Ray image will not be able to discriminate closer parts of the object from parts further away.

Prior art has already recognized the problem of determining the pose of an implant, to which United States patent U.S. Pat. No. 5,676,146 cites the solution of introducing a radiopaque marker element inside the implant, which marker element of course could be given any suitable shape. Such marker element may be a good solution for implants that themselves cause only slight absorption of X-Rays such as various organic resins, but for substances like stainless steel, the marker element would then usually be practically invisible. The usage of still heavier metals inside the implant in a shape without the symmetry would represent an overly complicated technology.

Furthermore, the inventor has recognized that earlier X-Ray methods have used two X-Rays with parallel detectors; however, the relation between effort involved and resulting quality has generally been unsatisfactory. In consequence, the inventor has recognized that there should be an improved method that requires relatively brief measurements only, and would rely on data processing propcedures for still attaining sufficient levels of accuracy and reliability.

### SUMMARY TO THE INVENTION

In consequence, amongst other things, it is an object of the present invention to provide a method for determining the pose of such implant that has a particular degree of symmetry, without necessitating specific shaping or manufacturing of the implant itself, but by relying on data processing procedures that have been taylored to the specific technical shape of the implant in question. Now therefore, according to one of its aspects, the invention is characterized according to the characterizing part of claim 1. The n-dimensional structure of symmetry may be an axis of rotary symmetry, a mirror plane for mirror symmetry, or a combination of the above in case of multiple symmetry.

The invention also relates to an apparatus being arranged for implementing the method as claimed in claim 1. Further advantageous aspects of the invention are recited in dependent claims.

### BRIEF DESCRIPTION OF THE DRAWING

These and further features, aspects and advantages of the invention will be discussed more in detail hereinafter with reference to the disclosure of preferred embodiments of the invention, and in particular with reference to the appended Figures that illustrate:

FIGS. 1 and 2, overall measuring arrangements for a single source position with respect to a mirror-symmetric implant object 26;

FIG. 3, an overall measuring arrangement for dual source positions with respect to the mirror-symmetric implant object 26;

FIGS. 4 and 5, overall measuring arrangements for a single source position with respect to a rotary-symmetric implant object 66;

FIG. 6, an overall measuring arrangement for dual source positions with respect to the rotary symmetric implant of FIGS. 4, 5;

FIG. 7, a flow chart embodiment of the procedures according to the invention.

### GENERAL CONSIDERATIONS FOR THE INVENTION

Generally, the position and orientation or pose of an implant with respect to an X-Ray imaging plane, such as a film or an image intensifier can be estimated by combining a CAD model of that implant with the projection image actually generated. However, in various situations where the implant has a certain degree of symmetry, the orientation so found may become undetermined, because there exists a further projection with an alternative orientation or pose that closely resembles the projection relating to the original orientation or pose. In case of a parallel projection, the two implant poses may even yield identical images. In practice, if the size of the implant is small relative to the source-image distance of the X-Rays, the projection can be approximated by a parallel projection.

Now, when the orientation of a symmetrical implant is estimated from an X-Ray image, the following alternative hypothesis will be calculated:

for a rotary symmetric model or implant, the symmetry axis is mirrored in a plane that is perpendicular to the viewing direction in order to obtaine the second, matching orientation. For a mirror symmetric model or implant, the normal of the symmetry plane is mirrored with respect to the viewing direction in order to obtain the second, matching orientation.

During a translation reconstruction run, successive overlapping images are recorded and the implant will therefore show on plural successive images. In these images, both the original implant orientation and also its alternative can be computed. Because of the mutual shifts between the images, the viewing direction will change slightly, just as will the alternative orientations. However, the “actual” orientation remains the same between successive images because there is only a translation and no rotation between successive images during the run.

### DETAILED DESCRIPTION OF PREFERRED EMBODIMENTS

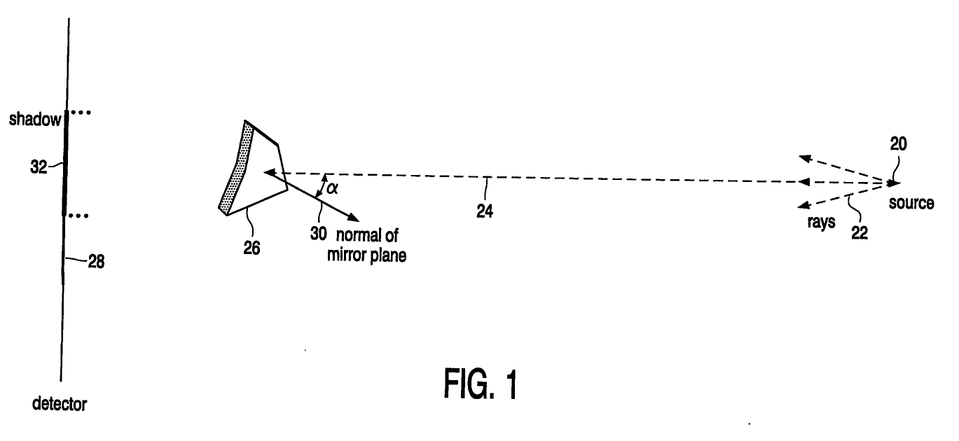

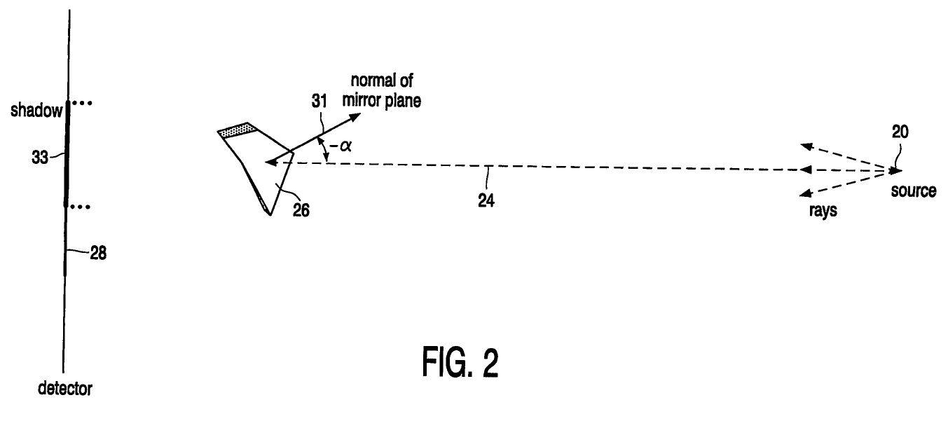

FIGS. 1 and 2 illustrate respective overall measuring arrangements for a single source position with respect to a mirror-symmetric implant object 26. In FIG. 1, source 20 emits a spreading beam of X-Rays 22 that is approximately rotary symmetric around axis 24, towards the implant 26. This beam will cause a shadow 32 of the implant object 26 to appear on detector imaging plane 28. Note that the detection proper has a two-dimensional organization, so that in effect a two-dimensional shadow will be detected, such as by an array of grey values. Now, the implant object 26 itself has been indicated in gray, and has a substantially flat and mirror symmetric shape. The thickness of this flat shape has been suggested by adding a shaded side in black. For objects with an appreciable thickness, the procedures recited hereinafter would remain the same. The normal to the mirror plane of flat shape 30 forms an angle +α with respect to the axis 24 of the X-Ray beam. The mirror plane itself has not been indicated, but it would be clear that axis 24 is not perpendicular to this mirror plane. If axis 24 would be perpendicular indeed, the problem considered here would not be present.

In FIG. 2, the relative orientation of implant object 26 has been mirrored with respect to the beam axis 24, the normal of the mirror plane now being labeled 31. The new orientation of the normal translates also into a changed appearance of the black shade of the implant object, and into a new projected angle of −α between normal 31 and axis 24. Due to the substantially parallel orientation of the X-Rays near the relatively small implant, the two shadows 32, 33 will be substantially identical. The inventor has recognized that this will render a determination of the pose of implant object 26, even as being based on the CAD model thereof and just one of these two orientations, either impossible or at least inaccurate. In fact, the outcome would readily produce either one of these two orientations as a result.

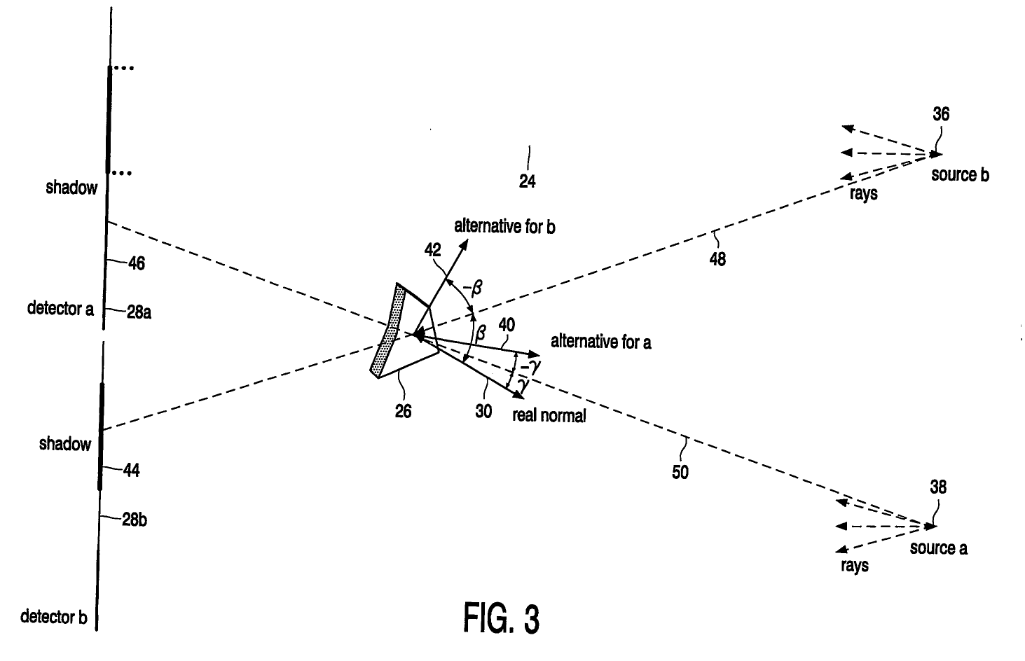

FIG. 3 illustrates an overall measuring arrangement for dual source positions 36, 38, with respect to the mirror-symmetric implant object 26 that has again been shown in the position of FIG. 1, together with its real normal 30. First source position 36 will produce shadow 44 on detector plane 28b, and lead to concluding of either real normal 30, or rather to concluding mirrored normal 42 of the implant object, again restricting to only the angle component in the plane of the Figure. The two orientations lie at angles ±β with respect to line 48 that connects the origin 36 of the source with roughly the center of gravity of shadow 44. Likewise, second source position 38 will produce shadow 46 on detector plane 28b, and lead to concluding of either real normal 30, or rather to concluding mirrored normal 40 of the implant object, again restricting as discussed above. The two orientations lie at angles ±γ with respect to line 50 that connects the origin 38 of the source with roughly the center of gravity of shadow 46. The above leads to unison among the two predicted poses of implant object 26. The person skilled in the art will know how to select a suitable displacement between source positions 36 and 38. A small value for the displacement will cause the two “alternatives” to lie close to each other, thereby rendering the the choice between the “real normal” and the “alternatives” more difficult. Other considerations would lead to choosing the two positions not too far from each other.

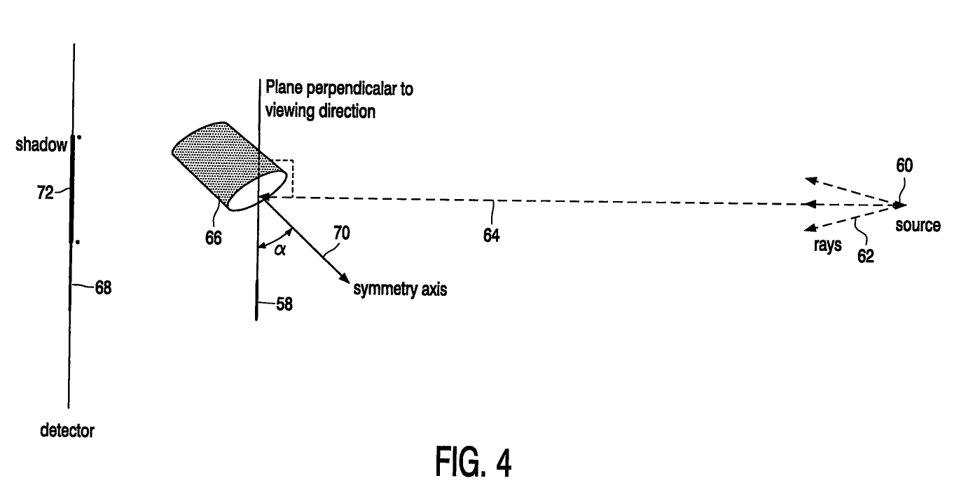

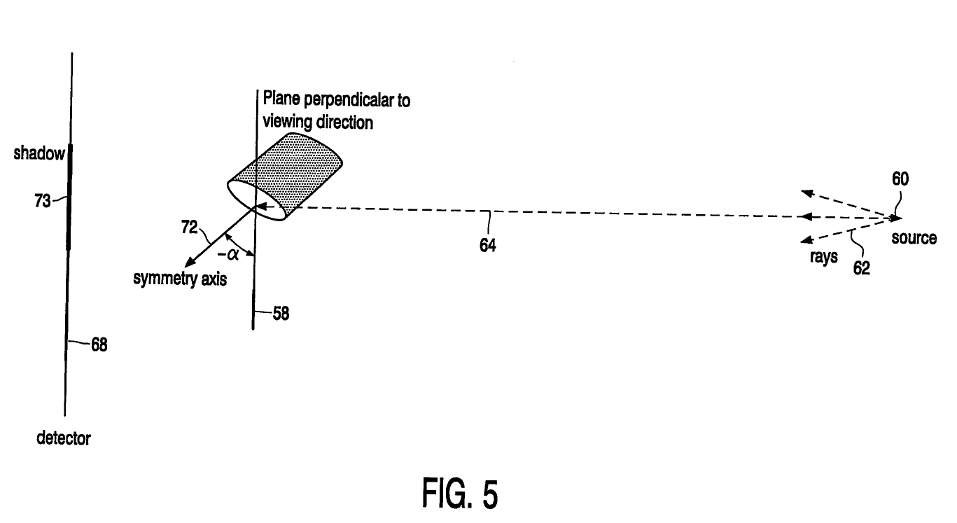

FIGS. 4 and 5 illustrate overall measuring arrangements for a single source position with respect to a rotary-symmetric implant object 66. It is understood that the implant would have no symmetry other than the rotary symmetry. In FIG. 4, source 60 emits a divergent beam of X-Rays 62 such as emitted by an approximately point-shaped source, along axis 64 towards the implant 66. This beam will cause a shadow 72 of the implant object 66 to appear on detector imaging plane 68. The detection again has a two-dimensional organization. Now, the implant object 66 itself has been indicated as a black cylinder with a grey end plane. The axis 70 of rotary symmetry forms an angle α with respect to the plane 58 that is perpendicular to the viewing direction, and which in the situation shown is both perpendicular to axis 64 and parallel to detector plane 68. The axis of symmetry need not lie in the plane of the drawing, but by itself this will not represent a problem. Note in particular, that the axes and also the labels of the various angles in this Figure are not related to those specified earlier with respect to FIGS. 1 and 2.

In FIG. 5, the relative orientation of implant object 66 has been mirrored with respect to the plane 58 that is perpendicular to the viewing direction, the axis of symmetry now being labeled 72. The new orientation leads to a new angle −α between plane 58 and symmetry axis 72. Due to the substantially parallel orientation of the X-Rays near the relatively small implant object, the two shadows 72, 73 will be substantially identical. The inventor has also in this case recognized that this will render a determination of the pose of implant object 66, even as based on the CAD model thereo and just one of these two orientations either impossible, or at best, inaccurate. In fact, either outcome would be equally probable.

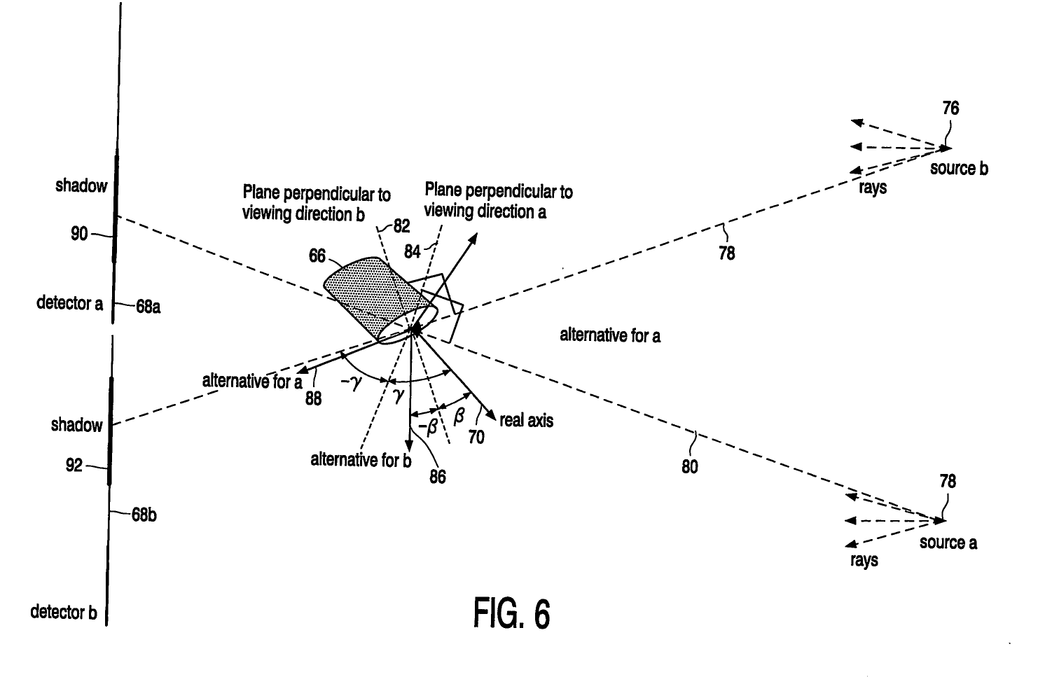

FIG. 6 illustrates an overall measuring arrangement for dual source positions 76, 78, with respect to the rotary symmetric implant object 66, that has again been shown in the position of FIG. 4, together with its real axis 70. First source position 76 will produce shadow 92 on detector plane 68b, and will lead to concluding of either real axis 70, or mirrored axis 68 of the implant object. The two orientations lie at angles ±α with respect to plane 82 that is perpendicular to line 78 which connects the origin 76 of the X-Ray source with roughly the center of gravity of shadow 92. Likewise, second source position 78 will produce a shadow 90 on detector plane 68b, and will lead to concluding of either real axis 70, or mirrored axis 88 of the implant object, again restricting to the plane of the drawing, as discussed above. Here, the two orientations lie at angles ±γ with respect to plane 84 that is perpendicular to line 80 which connects the origin of X-Ray source 78 with roughly the center of gravity of shadow 90. The above will again lead to unison among the two predicted poses of implant object 66. The person skilled in the art will know how to select a suitable displacement between source positions 76 and 78, generally as based on similar considerations as those given above with respect to FIG. 3.

Next to the cases of symmetry considered supra, the implant could also have both a rotary symmetry and also a mirror symmetry, such as when FIGS. 4 through 6 would indeed relate to a round bar, a double cone, or the like. In that case there would exist various mirrored poses that would result in the same shadow when taking only a single shadow. The problem is solved by taking the steps discussed above for the more simple cases of symmetry together, and then deciding among the various poses that were possible in principle. For example, three or even four X-Ray source positions could then be taken to reach the eventual decision.

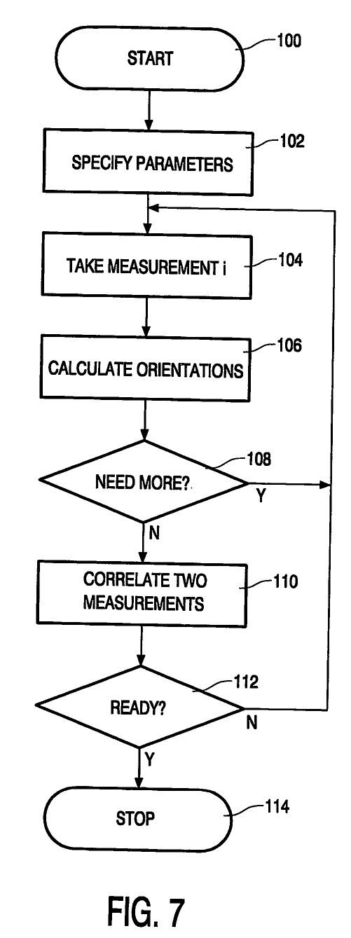

FIG. 7 illustrates a flow chart embodiment of the procedures according to the invention. Generally, the apparatus for manipulating the X-Ray source relative to the human or animal body under consideration can be conventional, and will in consequence not be discussed further. Also, such elements as are necessary to derive the shadows in the respective positions of the source relative to the body could be taken from an extensive patent and other litterature. Therefore, only the data processing procedure will be considered hereinafter. Now, in block 100, the necessary hardware and software facilities are assigned. In block 102, the necessary parameter values are specified, that will rate the distance from the source to the imaging plane, the two source positions with respect to each other, and possibly also with regard to an expected pose of the implant. For the latter, usually an approximate value will be known.

If also the best values for the measurements locations have to be found experimentally, this will be done by correlating the shadow properties along the translation direction of the source, supposing that the implant object remains stationary. This additional feature has not been considered in further detail herein, however.

Next, in block 104, a measurement #i is taken, either on-line, or off-line from a data base that had been produced earlier. From such measurement, in block 106, the possible orientations of the implant are calculated, taking into account the CAD model of the implant in question. In block 108, the system finds whether a further measurement result is necessary. If so (Y), revert to block 104. If such further measurement is not necessary (N), in block 110 two such measurements are correlated, which yields the orientation angle. If necessary, such correlation can be repeated with other measured values. If still further orientation information is necessary, the system reverts to block 104. If the orientation is not yet sufficiently known (N in block 112), the system reverts once more to block 104. If the outcome is sufficiently, however (Y in block 112), the system goes to block 114, whilst relinquishing assigned facilities as far as relevant.

The calculation as discussed above may restrict to just two measurements whilst making only one correlation, or to a larger number of measurements whilst using statistical procedures to attain smaller errors through limiting statistical spreads, and the like.

Now, the present invention has hereabove been disclosed with reference to preferred embodiments thereof. Persons skilled in the art will recognize that numerous modifications and changes may be made thereto without exceeding the scope of the appended claims. In consequence, the embodiments should be considered as being illustrative, and no restriction should be construed from those embodiments, other than as have been recited in the claims.

1. A method for determining a pose of an implant object that is located inside a human or animal body, on the basis of a CAD model of that implant object through a reconstruction X-Ray procedure viz à viz the implant object,
said method being characterized by comprising for an implant object that has a degree of symmetry according to an n-dimensional structure of symmetry the steps of:
generating a first measurement configuration and a second measurement configuration regarding an X-Ray source and a prespecified implant object position, and generating a first and a second implant shadow, respectively;
assuming for each said first and second measurement configuration an instance of said n-dimensional structure of symmetry;
calculating for each of said first and second measurement configuration from said shadow a pair of alternative poses of said implant object as being symmetrical with respect to said n-dimensional structure;
and finding among said pairs of alternative poses two matching poses that thereby produce an angle information with respect to said n-dimensional structure of symmetry of said implant object.

2. A method as claimed in claim 1, wherein said n-dimensional structure of mirror symmetry is a plane.

3. A method as claimed in claim 1, wherein said n-dimensional structure of symmetry is a straight line.

4. A method as claimed in claim 3, wherein said straight line is an axis of rotary symmetry of said implant object.

5. An apparatus being arranged for implementing a method as claimed in claim 1 for determining a pose of an implant object that is located inside a human or animal body, on the basis of a CAD model of that implant object through a reconstruction X-Ray procedure viz à viz the implant object,
said apparatus comprising for an implant object that has a degree of symmetry according to an n-dimensional structure of symmetry the following facilities:
a measuring facility for generating a first measurement configuration and a second measurement configuration regarding an X-Ray source and a prespecified implant object position, and for generating a first and a second implant shadow, respectively;
data processing means for through assuming for each said first and second measurement configuration an instance of said n-dimensional structure of symmetry and calculating for each of said first and second measurement configuration from said shadow a pair of alternative poses of said implant object as being symmetrical with respect to said n-dimensional structure;
and matching means for finding among said pairs of alternative poses two matching poses that thereby produce an angle information with respect to said n-dimensional structure of symmetry of said implant object.
